(19) United States
(12) Patent Application Publication
(10) Pub. No.: US 2026/0335641 A1
(43) Pub. Date: Oct. 29, 2026

(54) SYSTEM FOR GUIDING IMPLANTATION OF A LEAD OF AN IMPLANTABLE PULSE GENERATOR FOR TREATMENT OF A PELVIC FLOOR DYSFUNCTION AND/OR OF A PELVIC FLOOR-RELATED DISORDER

(71) Applicants: Universiteit Antwerpen, Antwerpen (BE); Universitair Ziekenhuis Antwerpen, Edegem (BE)

(72) Inventor: Stefan DE WACHTER, Wilrijk (BE)

(73) Assignees: Universiteit Antwerpen, Antwerpen (BE); Universitair Ziekenhuis Antwerpen, Edegem (BE)

(21) Appl. No.: 19/107,380

(22) PCT Filed: Aug. 28, 2023
(86) PCT No.: PCT/EP2023/073502

§ 371 Date: Feb. 27, 2025

(30) Foreign Application Priority Data
Aug. 30, 2022 (EP) 22192820.3

(51) Int. Cl. A61N 1/36; A61B 5/391; A61N 1/05

(52) U.S. Cl. CPC A61N 1/36007; A61B 5/391; A61N 1/0524; A61N 1/3606; A61B 2505/05

(57) ABSTRACT

A system comprising a processor wherein the processor is configured to: —receive measured data of the subject comprising: —pelvic floor muscle electromyography, PFM-EMG data of the subject, wherein the PFM-EMG data comprises at least: —a) action potential, AP, growth data, comprising one or more measurements of the peak-to-peak action potential growth, AP growth, of the subject, wherein the AP growth is an indication of an increase in contraction force per unit of stimulation delivered by the pulse generator (160); —determine from the measured data, real time guidance information for guiding a position and/or orientation of an electrode portion (126) during implantation, set of operating parameters of the pulse generator.

### FIELD OF THE INVENTION

The presently described system and methods are in the field of neuromodulation for treatment of a pelvic floor dysfunction and/or of a pelvic floor-related disorder.

### BACKGROUND OF THE INVENTION

Neuromodulation is useful for treatment of a pelvic floor dysfunction. A pelvic floor dysfunction is a cause of a pelvic floor-related disorder including overactive bladder, a non-obstructive urinary retention, faecal incontinence, pain (e.g. pelvic, rectal, anal, bladder), sexual dysfunction (e.g. erectile dysfunction, female sexual dysfunction). Neuromodulation involves implantation of a neurostimulator having a lead and pulse generator, wherein electrode contacts of the lead are positioned adjacent to a sacral nerve or pudendal root of the subject.

During implantation, the neurostimulator lead containing a plurality of electrode contacts is placed through the third or fourth sacral foramen and is connected to a pulse generator enabling stimulation of the sacral spinal nerves. The placement of the lead occurs under general or local anaesthesia and correct lead position with respect to the sacral spinal nerves is determined primarily by medical imaging. Afterwards a set of operating parameters of the pulse generator electrodes is set; this includes one or more stimulating and reference electrode(s) are assigned to the lead contacts (and optionally housing).

If, sometime after implantation of the neurostimulator the patient experiences a loss of efficacy or an undesirable electrical stimulation (e.g., painful stimulation) the operating parameters are changed, meaning the previously selection of stimulating contacts are changed and new stimulating and/or reference electrode(s) are assigned to the lead electrodes.

US 2016/0114167 A1 discloses sacral modulation for treatment of over active bladder (OAB), and electromyography (EMG) to assist with lead placement, however, the methods described using EMG peak-to-peak compound muscle action potential (CMAP) until an EMG response is observed, which method still results in non-responders.

Neuromodulation fails in a proportion of patients, due to inadequate electrode placement with suboptimal coupling of the electrode and nerve, and/or due suboptimal selection of set of operating parameters including of the lead stimulating contacts. The present invention overcomes problems of the art.

### SUMMARY OF THE INVENTION

Provided herein is a system for guiding implantation of an electrode portion (126) of a lead (120) of a neurostimulator for treatment of a pelvic floor dysfunction and/or of a pelvic floor-related disorder of a subject, the system comprising a processor wherein the processor is configured to:
receive measured data of the subject comprising:
   pelvic floor muscle electromyography, PFM-EMG data of the subject, wherein the PFM-EMG data comprises:
      a) action potential, AP, growth data, comprising one or more measurements of the peak-to-peak action potential growth, AP growth, of the subject, wherein the AP growth is an indication of an increase in contraction force per unit of stimulation delivered by the pulse generator (160);
      and optionally b) motor threshold, MT, data comprising one or more measurements of the motor threshold (MT) of the subject, where the MT is an indication of a minimum stimulation amplitude delivered by a pulse generator (160) of the neurostimulator that induces a measurable action potential in the pelvic floor of the subject as measured by the PFM-EMG;
      and optionally c) peak-to-peak amplitude of the action potential, PtP-AP, data comprising one or more measurements of the PtP-AP of the subject, wherein the PtP-AP is an indication of a contraction force of the pelvic floor as measured by the PFM-EMG during stimulation delivered by the pulse generator (160);
determine from the measured data, real time guidance information for guiding a position and/or orientation of an electrode portion (126) during implantation.

Provided herein is a system for guiding implantation of an electrode portion (126) of a lead (120) of a neurostimulator for treatment of a pelvic floor dysfunction and/or of a pelvic floor-related disorder of a subject, the system comprising a processor wherein the processor is configured to:
receive measured data of the subject comprising:
   pelvic floor muscle electromyography, PFM-EMG data of the subject, wherein the PFM-EMG data comprises one or more of:
      a) motor threshold, MT, data comprising one or more measurements of the motor threshold (MT) of the subject, where the MT is an indication of a minimum stimulation amplitude delivered by a pulse generator (160) of the neurostimulator that induces a measurable action potential in the pelvic floor of the subject as measured by the PFM-EMG;
      b) peak-to-peak amplitude of the action potential, PtP-AP, data comprising one or more measurements of the PtP-AP of the subject, wherein the PtP-AP is an indication of a contraction force of the pelvic floor as measured by the PFM-EMG during stimulation delivered by the pulse generator (160);
      c) action potential, AP, growth data, comprising one or more measurements of the peak-to-peak action potential growth, AP growth, of the subject, wherein the AP growth is an indication of an increase in contraction force per unit of stimulation delivered by the pulse generator (160);
determine from the measured data, real time guidance information for guiding a position and/or orientation of an electrode portion (126) during implantation.

Provided herein is a system for adjusting and/or determining a set of operating parameters of a pulse generator (160) of a neurostimulator for treatment of a pelvic floor dysfunction and/or of a pelvic floor-related disorder in a subject, the system comprising a processor wherein the processor is configured to:
receive measured data of the subject comprising:
   pelvic floor muscle electromyography, PFM-EMG data of the subject, wherein the PFM-EMG data comprises:
      a) motor threshold, MT, data comprising one or more measurements of the motor threshold (MT) of the subject, where the MT is an indication of a minimum stimulation amplitude delivered by a pulse generator (160) of the neurostimulator that induces a measurable action potential in the pelvic floor of the subject as measured by the PFM-EMG;
      b) peak-to-peak amplitude of the action potential, PtP-AP, data comprising one or more measurements of the PtP-AP of the subject, wherein the PtP-AP is an indication of a contraction force of the pelvic floor as measured by the PFM-EMG during stimulation delivered by the pulse generator (160);
      c) action potential, AP, growth data comprising one or more measurements of the peak-to-peak action potential growth, AP growth, of the subject, wherein the AP growth is an indication of an increase in contraction force per unit of stimulation delivered by the pulse generator (160);
determine from the measured data, a set of operating parameters of the pulse generator.

Provided herein is a system for adjusting and/or determining a set of operating parameters of a pulse generator (160) of a neurostimulator for treatment of a pelvic floor dysfunction and/or of a pelvic floor-related disorder in a subject, the system comprising a processor wherein the processor is configured to:
receive measured data of the subject comprising:
   pelvic floor muscle electromyography, PFM-EMG data of the subject, wherein the PFM-EMG data comprises:
      a) action potential, AP, growth data comprising one or more measurements of the peak-to-peak action potential growth, AP growth, of the subject, wherein the AP growth is an indication of an increase in contraction force per unit of stimulation delivered by the pulse generator (160);
      and optionally b) motor threshold, MT, data comprising one or more measurements of the motor threshold (MT) of the subject, where the MT is an indication of a minimum stimulation amplitude delivered by a pulse generator (160) of the neurostimulator that induces a measurable action potential in the pelvic floor of the subject as measured by the PFM-EMG;
      and optionally c) peak-to-peak amplitude of the action potential, PtP-AP, data comprising one or more measurements of the PtP-AP of the subject, wherein the PtP-AP is an indication of a contraction force of the pelvic floor as measured by the PFM-EMG during stimulation delivered by the pulse generator (160);
determine from the measured data, a set of operating parameters of the pulse generator.

An MT that is lowest may be indicative of a therapeutically effective pose of the electrode portion (126) and/or or a therapeutically effective set of operating parameters, or
an MT that is low is indicative of a therapeutically effective pose of the electrode portion (126) and/or or a therapeutically effective set of operating parameters, wherein a low MT is equal to or below 2 mA.

An MT that is lowest or low may be indicative of a therapeutically effective pose of the electrode portion (126) and/or or a therapeutically effective set of operating parameters, wherein a low MT is equal to or below 2 mA.

A PtP-AP that is highest may be indicative of a therapeutically effective pose of the electrode portion (126) and/or or a therapeutically effective set of operating parameters, or
a high PtP-AP is indicative of a therapeutically effective pose of the electrode portion (126) and/or or a therapeutically effective set of operating parameters, wherein a high PtP-AP is equal to or above 20 μV, preferably equal to or above 50 μV.

A PtP-AP that is highest or high may be indicative of a therapeutically effective pose of the electrode portion (126) and/or or a therapeutically effective set of operating parameters, wherein a high PtP-AP is equal to or above 20 μV, preferably equal to or above 50 μV.

An AP growth that is highest is indicative of a therapeutically effective pose of the electrode portion (126) and/or or a therapeutically effective set of operating parameters, or
a high AP growth is indicative of a therapeutically effective pose of the electrode portion (126) and/or or a therapeutically effective set of operating parameters, wherein a high AP growth has an initial AP growth gradient of ≥0.4, preferably ≥0.4.

An AP growth that is highest or high may be indicative of a therapeutically effective pose of the electrode portion (126) and/or or a therapeutically effective set of operating parameters, wherein a high AP growth has a ratio (PtP-AP at a stimulation amplitude greater than MT)/(PtP-AP at a stimulation amplitude of MT) greater than 1.

The measured data may further comprise self-observation data of the subject, wherein the self-observation data comprises:
d) sensory threshold, ST, data comprising one or more measurements of the ST of the subject, wherein the ST is an indication of a minimum stimulation amplitude delivered by the pulse generator that induces a physical sensation felt by the subject; and
e) location of sensation, LoS, data comprising one or measurements of the LoS of the subject data, wherein the LoS is an indication by the subject of a location of the feeling of the physical sensation in the body caused by stimulation delivered by the pulse generator.

A MT:ST ratio indicating that ST is greater than MT is indicative of a therapeutically effective position and orientation of the electrode portion (126) and/or of a therapeutically effective set of operating parameters.

A LoS contained within an anal region or perianal region or genital region of the subject, is indicative of a therapeutically effective position and orientation of the electrode portion (126) and/or of a therapeutically effective set of operating parameters.

The d) ST data and the e) LoS data may be collected while the subject is conscious.

The a) AP growth data, optionally the b) MT data and optionally the c) PtP-AP data may be collected while the subject is conscious or unconscious.

The measured data may comprise a) the AP growth data, b) the MT data, c) the PtP-AP data, d) the ST and e) the LoS data.

The a) AP growth data, b) MT data, the c) PtP-AP data, may be collected while the subject is conscious or unconscious.

The set of operating parameters may comprise one or more of:
a selection of contacts in the electrode portion (126) (contact configuration parameter); and
a stimulation amplitude parameter.

The measured data may be collected while the electrode portion (126) of the lead is being positioned towards or a is placed adjacent to a sacral or pudendal root of the subject.

The pelvic floor-related disorder may be one or more of an overactive bladder, non-obstructive urinary retention, faecal incontinence, pain (e.g. pelvic, rectal, anal, bladder), sexual dysfunction (e.g. erectile dysfunction, female sexual dysfunction).

Provided herein is a computer program comprising instructions which, when the program is executed by a computer, cause the computer to carry out the steps recited herein.

Provided herein is a computer-readable medium comprising instructions which, when the program is executed by a computer, cause the computer to carry out the steps recited herein.

### BRIEF DESCRIPTION OF THE FIGURES

FIG. 1 is a schematic view of an electrode portion of a multi-contact lead.

FIG. 2 is a schematic view of an electrode portion of a single-contact lead.

FIG. 3 is a schematic view of a pulse generator.

FIG. 4 is a view of the pelvic floor area (202) showing various regions.

FIG. 5 is a view of the pelvic floor area, and showing the anus and female genitals.

FIG. 6 is a view of the pelvic floor area, and showing the anus and male genitals.

FIG. 7 is an example of a reference illustration, showing the pelvic floor area.

FIG. 8 is an example of a reference illustration, showing the pelvic floor area, and midline and band.

FIG. 9 is an example of a reference illustration, showing the pelvic floor area, and anal, perianal, genital and other zones, wherein the anal and perianal zones are nested.

FIG. 10 is an example of a reference illustration, showing the pelvic floor area, and anal, perianal, genital and other zones, wherein the anal, perianal, genital zones are nested.

FIG. 11 is an example of a reference illustration, showing the pelvic floor area, showing a plurality of zones arranged in a grid.

FIG. 12 graphs A to G show AP growth curves for patients whose treatment by neuromodulation failed (non-responders) or succeeded (responders) for separate PFM-EMG probe locations A to G. Graphs A to C are at different measurement depths (A superficial; B centre; C deep). Graphs D to G are at different sides (D anterior; E contralateral; F ipsilateral; G posterior)

FIG. 13 is an AP growth curve combining the AP growth curves of FIG. 12 graphs A to G.

### DETAILED DESCRIPTION OF THE INVENTION

Before the present method and products of the invention are described, it is to be understood that this invention is not limited to particular methods, components, products or combinations described, as such methods, components, products and combinations may, of course, vary. It is also to be understood that the terminology used herein is not intended to be limiting, since the scope of the present invention will be limited only by the appended claims.

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. It will be appreciated that the terms “comprising”, “comprises” and “comprised of” as used herein comprise the terms “consisting of”, “consists” and “consists of”.

The recitation of numerical ranges by endpoints includes all numbers and fractions subsumed within the respective ranges, as well as the recited endpoints.

The term “about” or “approximately” as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” or “approximately” refers is itself also specifically, and preferably, disclosed.

Whereas the terms “one or more” or “at least one”, such as one or more or at least one member(s) of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members.

All references cited in the present specification are hereby incorporated by reference in their entirety. In particular, the teachings of all references herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination.

In the present description of the invention, reference is made to the accompanying drawings that form a part hereof, and in which are shown by way of illustration only of specific embodiments in which the invention may be practiced. Parenthesized or emboldened reference numerals affixed to respective elements merely exemplify the elements by way of example, with which it is not intended to limit the respective elements. Unless otherwise indicated, all figures and drawings in this document are not to scale and are chosen for the purpose of illustrating different embodiments of the invention. In particular the dimensions of the various components are depicted in illustrative terms only, and no relationship between the dimensions of the various components should be inferred from the drawings, unless so indicated.

The terms “distal” or “distal to” and “proximal” or “proximal to” are used throughout the specification, and are terms generally understood in the field to mean towards (proximal) or away (distal) from the operator's side of device (e.g. EMG probe). Thus, “proximal” or “proximal to” means towards the operator's side and, therefore, away from the patient's side. Conversely, “distal” or “distal to” means towards the patient's side and, therefore, away from the operator's side.

The term “pose” refers to a position and orientation within the body. It is used in particular in reference to the electrode portion of the lead.

The term “stimulation” refers to electrical stimulation.

The ventral (V) direction is a term known in the art, and refers to a front (face) direction of the subject. The dorsal (D) direction is a term known in the art, and refers to a rear (back) direction of the subject. The lateral (L) direction is a term known in the art, and refers to a sideways (left to right, or vice versa) direction of the subject. The cranial direction is a term known in the art, and refers to an upward (towards the head) direction of the subject. The caudal direction is a term known in the art, and refers to a downward (towards the feet) direction of the subject.

The present invention is based on the finding that measured data of the subject comprising pelvic floor muscle electromyography, PFM-EMG, data of the subject of the subject can be used to guide a position and orientation of a neurostimulator lead during lead implantation, and/or used to adjust and/or determine a set of operating parameters of a neurostimulator.

A neurostimulator is known the art. It comprises a pulse generator and one or more leads. The neurostimulator is implantable. The neurostimulator is for sacral nerve or pudendal root neuromodulation. The neurostimulator is for treatment of pelvic floor dysfunction and/or a pelvic floor-related disorder in a subject.

The pulse generator is configured to generate electrical stimulation. An exemplary pulse generator (160) is shown in FIG. 3. It typically comprises a controller (162) (e.g. microprocessor, memory), circuitry (164) for generating the electrical pulses, and a power source (164) (e.g. rechargeable battery). Other components include an antenna (167) for wireless exchange of data and/or wireless charging, and a connector block (168) for electrical and mechanical connection to the lead (120). The pulse generator generates pulses according to a set of operating parameters that includes electrical contact selection, and stimulation amplitude.

The operating parameters are usually read and processed by the controller. The pulse generator may have a housing (169) having an exterior surface that is electrically conducting (e.g. at least partially metallic); in some configurations the electrically conducting housing acts as a reference electrode and the other stimulation electrode is located in at least one electrical contact of the lead (unipolar stimulation). The pulse generator may have housing having an exterior surface that is non-electrically conducting; usually, both reference and stimulation electrodes are located in two or more of the electrical contacts of the lead (bipolar stimulation).

The pulse generator may be one channel or multi-channel. A one channel pulse generator is configured to deliver electrical stimulation to only one pair of contacts (non-switchably). A multi-channel pulse generator is configured to deliver electrical stimulation switchably to different pairs of contacts where at least one contact of the pair is different.

Pulse generators are preferably small to limit patient trauma and discomfort. The pulse generator is implantable. The pulse generator described herein is for sacral neuromodulation or for pudendal neuromodulation. The pulse generator may be for treatment of pelvic floor dysfunction and/or a pelvic floor-related disorder in a subject.

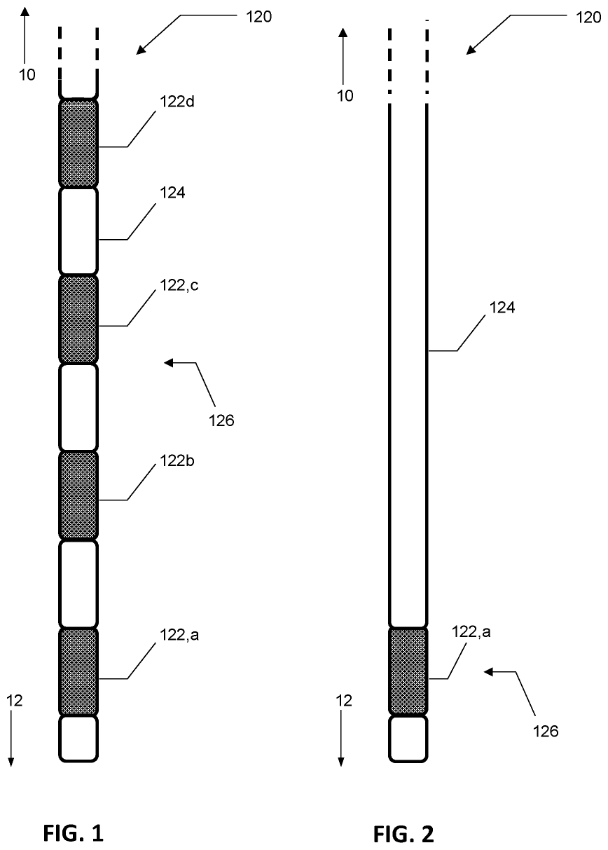

An exemplary lead is shown in FIGS. 1 and 2. The lead (120) comprises a longitudinal lead shaft (124) having a proximal end (10) and a distal end (12). At the distal end (12) an electrode portion (126) is disposed with at least one electrical contact (122, a to d). An electrical contact (122, a to d), also known as a contact herein, is electrically conductive and configured for electrical contact with the tissue to be stimulated. The shaft (124) may contain a lumen (e.g. for receiving a stylet to stiffen the lead during placement) or may be lumen-less.

The pulse generator delivers stimulation to an electrode pair, wherein one electrode is a stimulation electrode and the other electrode is a reference electrode. The reference electrode may be a housing of the pulse generator or may be a contact (122, a to d) of the electrode portion (126). The stimulation electrode is one or more contacts (122, a to d) of the electrode portion (126).

The electrode portion may comprise only one electrical contact (when the pulse generator housing acts as a reference electrode). A one contact lead is exemplified in FIG. 2. The electrode portion may comprise at least two electrical contacts (one or more contacts (122, a to d) may be a reference electrode, or the pulse generator housing may act as a reference electrode). A four contact lead is exemplified in FIG. 1. Where the electrode portion comprises at least two electrical contacts, they are spatially and electrical separated longitudinally along and/or circumferentially around the lead shaft. The number of contacts is preferably 4 or more. The number of leads of the neurostimulator may be one, or two or more, preferably only one. The one or more leads are implantable. The one or more leads is for sacral neuromodulation or pudendal root neuromodulation. The one or more leads may be for treatment of pelvic floor dysfunction and/or of pelvic floor-related disorder in a subject.

The measured data is collected while the electrode portion (126) of the lead is being positioned towards or is placed adjacent to a sacral or pudendal root of the subject.

The electrode portion of the lead is ideally positioned adjacent to a sacral nerve of the subject or adjacent to a pudendal root of the subject. In these positions, the effect is a neuromodulation for treatment of dysfunction of the pelvic floor, or for treatment of a pelvic floor-related disorder. During placement, the electrode portion is navigated towards these positions, usually under primary guidance of medical imaging (e.g. X-ray imaging). With the presently-described system, the pose of the electrode portion can be adjusted by the practitioner during implantation based on the measured data to provide are more effective treatment.

Pelvic floor muscle (PFM) electromyography (EMG) (PFM-EMG) is known in the art. PFM-EMG measurements are made using an PFM-EMG probe configured for capturing PFM measurements. The PFM-EMG probe is configured for positioning intravaginally (in women) or intrarectally (in women or men). The PFM-EMG probe is a surface contact EMG probe. The PFM-EMG probe may contain multiple electrical contacts for making multiple electrical connections to a tissue surface. The multiple electrical contacts may be disposed on a rigid (non-compressible) body. Examples of suitable PFM-EMG probes include those manufactured by Novuqare Pelvic Health BV (NL) (MAPLe probe), Neen UK (UK) (Periform intravaginal surface EMG probe).

The measured data of the subject contains data measured from the subject while the pulse generator sends stimulating pulses to the lead. The measured data comprises PFM-EMG data of the subject and optionally self-observation data of the subject. The measured data may be measured during implantation. The measured data may be measured for a pre-implanted neurostimulator after implantation (e.g. a first time set up and optimisation). The measured data may be measured for a pre-implanted neurostimulator in response (e.g. some time after implantation (e.g. months or years), and after the neurostimulator had previously higher efficacy).

The measured data may be recorded at different poses (different position and/or orientation) of the electrode portion of the lead within the body of the subject. This may be done by steering the lead, in particular the electrode portion, into different poses during implantation. At each pose, the measured data is recorded. The measured data can guide the practitioner towards a therapeutically effective pose of the lead. One or more (e.g. all of the) contacts of the electrode portion may deliver the stimulation at each pose when guiding the pose. Indicators of a therapeutically effective pose of the electrode portion, based on the measured data, are described elsewhere herein.

The measured data may be recorded at different selections of contact(s) of the electrode portion. The selection of contact(s) refers to the electrode pair (reference and stimulation contact(s)) selected that is used to deliver the stimulation. At each contact selection, the measured data is recorded. The measured data can guide the practitioner towards a therapeutically effective set of operating parameters of the pulse generator. Indicators of a therapeutically effective set of operating parameters, based on the measured data, are described elsewhere herein. At each contact selection, the subject indicates the location in the body where a physical sensation, caused by the stimulation, is felt. The lead may be pre-implanted. The lead may be being implanted.

The PFM-EMG data is determined from the PFM-EMG measurements collected while the pulse generator sends stimulating pulses to the lead. The EMG data may be collected while the subject is conscious (e.g. awake, under local anaesthesia) or unconscious (e.g. sleeping, under general anaesthesia).

The PFM-EMG data may be determined during implantation. The PFM-EMG data may be determined at different poses of the electrode portion of the lead. The PFM-EMG data may be determined for a pre-implanted neurostimulator after implantation (e.g. a first time set up and optimisation). The PFM-EMG data may be determined for a pre-implanted neurostimulator in response (e.g. some time after implantation (e.g. months or years), and after the neurostimulator had previously higher efficacy). The PFM-EMG data may be determined at different selections of contact(s) of the electrode portion. The PFM-EMG data comprises one or more of motor threshold, MT, data (MT data), peak-to-peak amplitude of the action potential data (PtP-AP data), and action potential growth data (AP growth data).

The MT data comprises one or more measurements of the motor threshold (MT) of the subject. The MT is an indication of a minimum stimulation amplitude delivered by the pulse generator through the lead that induces a measurable action potential in the pelvic floor of the subject as measured by PFM-EMG. To measure MT, the stimulation amplitude delivered by the pulse generator to the lead is varied (e.g. gradually increased or decreased) to find the minimum stimulation amplitude at which action potential is detected by the PFM-EMG. The stimulation amplitude may be increased, and the MT is the stimulation amplitude at which an action potential is first detected by the PFM-EMG. The stimulation amplitude may be decreased, and the MT is the stimulation amplitude at which the action potential ceases to be detected by the PFM-EMG. The MT values obtained by increasing or decreasing the stimulation amplitude are the same or similar. An average may be taken of the MT values obtained by increasing or decreasing the stimulation amplitude.

The MT is indicative of the minimum stimulation amplitude delivered by the pulse generator that can induce the action potential. The MT is typically a scalar value. It may be expressed in as a current (e.g. mA) or voltage (e.g. mV). PFM-EMG data may contain one or multiple MT measurements.

PtP-AP data comprises one or more measurements of the Peak-to-Peak amplitude of the Action Potential (PtP-AP) of the subject. Peak to peak amplitudes is understood in the art as meaning amplitude between adjacent an peak high and peak low. The PtP-AP is an indication of a contraction force of the pelvic floor at the MT as measured by the PFM-EMG.

To measure PtP-AP, stimulation is delivered by the pulse generator to the lead at a stimulation amplitude equal to the measured MT±20%, more preferably MT±10%, even more preferably MT±5%, and the action potential in the pelvic floor of the subject is measured by the PFM-EMG at the MT stimulation amplitude.

The PtP-AP is typically a scalar value. It may be expressed as a voltage (e.g. V). PFM-EMG data may contain one or multiple PtP-AP measurements.

AP growth data comprises one or more measurements of the peak-to-peak action potential growth (AP growth) of the subject. The AP growth is an indication of the change in contraction force per unit of stimulation by the pulse generator.

To measure AP growth, stimulation delivered by the pulse generator to the lead is varied (e.g. gradually increased or decreased) within a range having a lower limit equal to or greater than the MT, and the action potential in the pelvic floor of the subject is measured by the PFM-EMG at each stimulation amplitude. A value representative of a correlation between the amplitude of the pulse generator stimulation and the measured action potential (PtPAP), or a curve (AP growth curve) showing amplitude of the pulse generator stimulation and the measured action potential (Pt-PAP) provides the AP growth of the subject. The AP growth may be a scalar value. PFM-EMG data may contain one or multiple AP growth measurements.

AP growth may be an AP growth value that is:
(PtPAP at a stimulation amplitude greater than MT)/(PtPAP at a stimulation amplitude of MT),
   wherein PtP-AP is the measured peak-to-peak action potential growth of the subject.

AP growth may be AP growth curve is a curve on a graph showing PtPAP (in μV, mV, μA or mA) on one axis (typically y-axis) and stimulation amplitude (in μV, mV, μA or mA depending on if the stimulator is constant voltage (V) or constant current (A) controlled) on the other axis (typically x-axis). The lowest value of the stimulation amplitude is typically a value of MT in the range MT to (MT+(10% of MT)).

AP growth may be an AP growth gradient. An AP growth gradient is a gradient of an AP growth curve resulting from multiple measurements of PtPAP at different values of stimulation amplitude, MT+n, for instance,
(PtPAP) vs stimulation amplitude (MT+n)
   wherein PtP-AP is the measured peak-to-peak action potential growth of the subject at the motor threshold (MT+n), and n is a value of a change (increment or decrease) in the MT. The y axis may be PtPAP (in μV, mV, μA or mA) and the x-axis may be stimulation amplitude, MT+n (in μV, mV, μA or mA depending on if the stimulator is constant voltage (V) or constant current (A) controlled); the gradient is typically Δy/Δx. The values of n are greater than or equal to 0. The gradient may be determined, for example, by curve fitting and determining a gradient of a linear (growth) part of the fitted curve, or by determining the gradient between two points within a linear (growth) part of the AP growth curve.

AP growth is preferably an initial AP growth gradient that is an indication of a gradient between two points in an initial portion (stimulation amplitude, x-axis) of the AP growth curve. The first point (P1) of the two points has a stimulation amplitude between MT and (MT+(10% of MT)). The second point (P2) has a stimulation amplitude greater than (MT+(10% of MT)) and less than or equal to (MT+(50% of MT)). A separation between P1 and P2 is preferably at least (5% of MT). The initial AP growth gradient may be expressed as:

((Pt-PAP at P2)−(Pt-PAP at P1))/((stimulation amplitude at P2)−(stimulation amplitude at P1)).

The self-observation data is collected while the subject is conscious. The self-observation data is collected while the pulse generator sends stimulating pulses to the lead. The self-observation data may be collected at different poses of the electrode portion of the lead during implantation. The PFM-EMG data may be collected at different selections of contact(s) of the electrode portion. It is noted that the self-observation data is essentially consistent for each patient, for example, the same self-observation data is typically obtained for the same patent recorded on different occasions e.g. different times, on different days.

The self-observation data comprises sensory threshold, ST, data comprising one or more measurements of the ST of the subject. The ST is an indication of a minimum stimulation amplitude delivered by the pulse generator that induces a physical sensation felt by the subject.

To measure ST, the stimulation amplitude delivered by the pulse generator to the lead is varied (e.g. gradually increased) until the subject feels a physical sensation. The location of the feeling might be in the perineal region when the lead is well placed. The location of the feeling might be in the back or in the legs when the lead is poorly placed. The ST is indicative of the minimum stimulation amplitude delivered by the pulse generator that can be felt by the subject. Typically, all the lead contacts are selected stimulation to deliver stimulation. The ST is typically a scalar value. It may be expressed as a current (e.g. mA) or voltage (e.g. mV). The self-observation data may contain one or multiple ST measurements.

The self-observation data comprises location of sensation, LoS, data comprising one or measurements of the LoS of the subject. The LoS is an indication by the subject of a location of the feeling of the physical sensation caused by stimulation delivered by the pulse generator. To measure LoS, the stimulation is delivered by the pulse generator to the lead at an amplitude greater than the ST.

During measurement, pose of the electrode portion may be adjusted. This may be done by steering the lead into a different position and/or orientation during implantation. At each pose, the subject indicates (by LoS measurement) the location in the body where a physical sensation, caused by the stimulation, is felt.

During measurement, different contacts of the electrode may be selected to deliver the stimulation, each contact having a different contact position on the lead. At each contact position, the subject indicates (by LoS measurement) the location in the body where a physical sensation, caused by the stimulation, is felt. The lead may be pre-implanted. The lead may be being implanted.

The LoS may be expressed as an alphanumeric value or co-ordinates representative of the location.

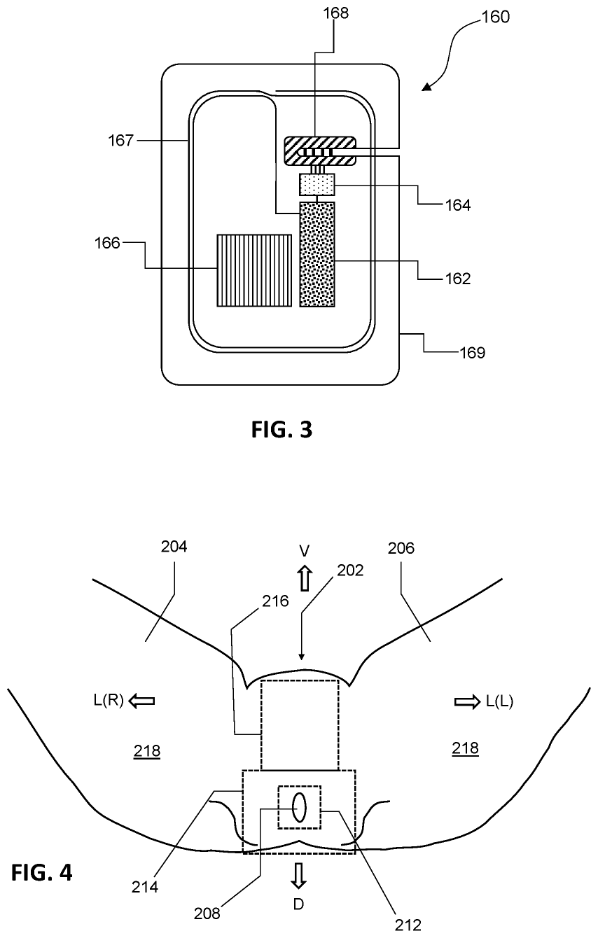

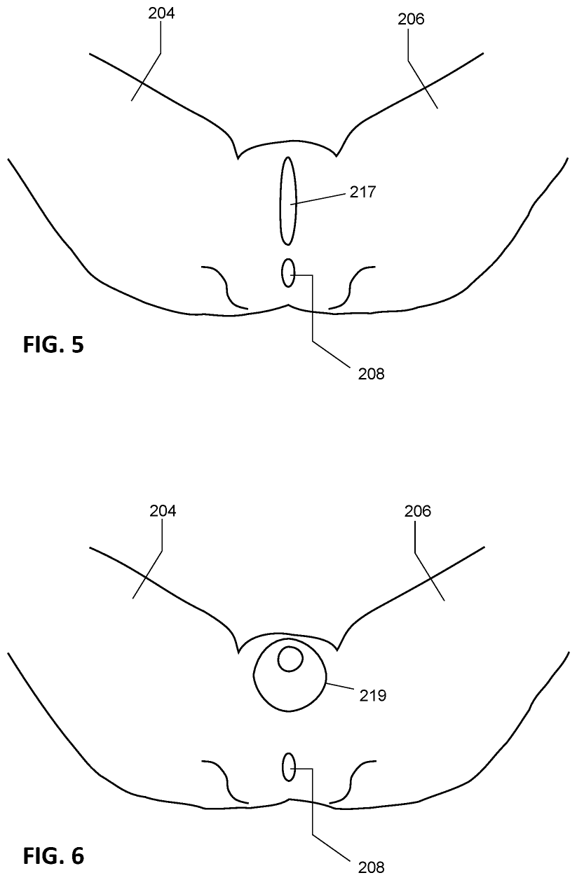

The sensation may be located in a region of the body, in particular of the pelvic floor area or outside the pelvic floor area. Exemplary regions are highlighted in FIG. 4, depicting a view of the pelvic floor area (202) which view is from a caudal to cranial direction. Shown are an anus (208), an anal region (212), a perianal region (214), a genital region (216) and other region (218). Also shown are the right (204) and left (206) legs. For clarity the genitals have not been depicted; these are indicated in FIG. 5 showing female genitals (217), and in FIG. 6 (219) showing the male genitals.

The anal region (212) encompasses the anus (208), and does not include the perianal region (212), the genital region (216) and the other region (218). The anal region (212) may coincide with the dermatome S5.

The perianal region (214) encompasses the perianal, and does not include anal region (212), the genital region (216) and other region (218). The perianal region (214) may coincide with the dermatome S4.

The genital region (216) encompasses the genitals (217, 219), and does not include anal region (212), the perianal region (212) and other region (218). The genital region (216) may coincide with the dermatome S3.

A perineal region is a combination of the perianal region (214) and genital region (216).

The other region (218) does not include anal region (210), the perianal region (212), the genital region (216) and other region (218). It can encompass a leg (204, 206), toe, lower back.

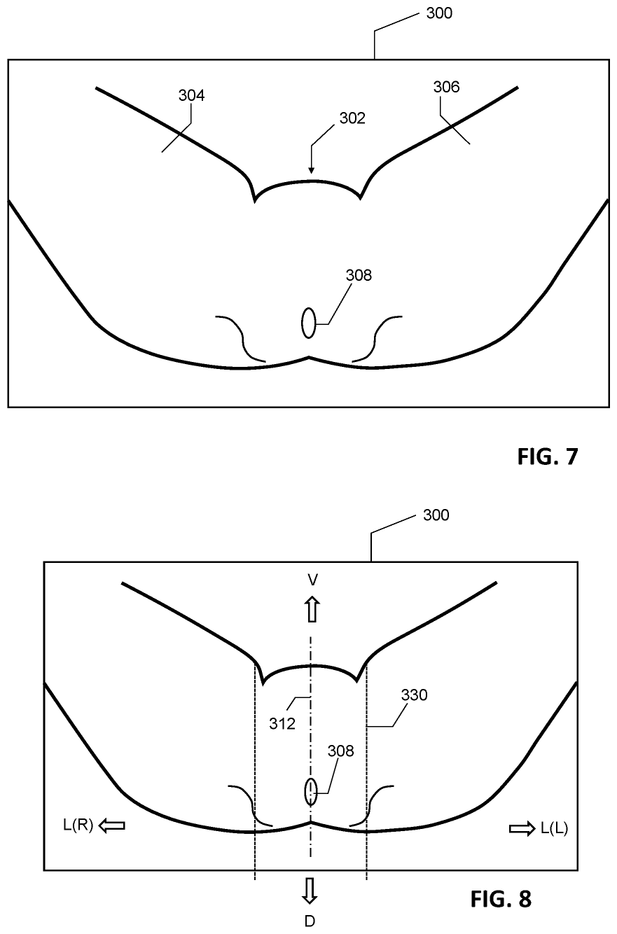

The LoS is representative of a region of the body where the physical sensation is felt. The LoS may be indicated on or with reference to a reference illustration of an exterior of a body, in particular of the exterior of the body in the area of the pelvic floor. The area of the pelvic floor is shown in the reference illustration in inferior (feet) to superior (head) direction. The reference illustration is preferably a generalised (non-subject) illustration of the exterior of the pelvic floor area. The reference illustration may be provided on a printable medium (e.g. paper, card, cardboard, hardboard). The reference illustration may be provided on a display e.g. computer screen display, touch screen display (e.g. on a smart device, smart phone, tablet). An example of a reference illustration (300) is shown in FIG. 7, showing a reference illustration of the pelvic floor area (302), anus (308), right (204) and left (206) legs. The reference illustrations of FIGS. 8 to 11 are based on FIG. 7. The indication may be provided by the subject by marking the printable medium (e.g. with a writing instrument such as a pen, pencil, printable medium marker), or by touching a zone on a touch screen display.

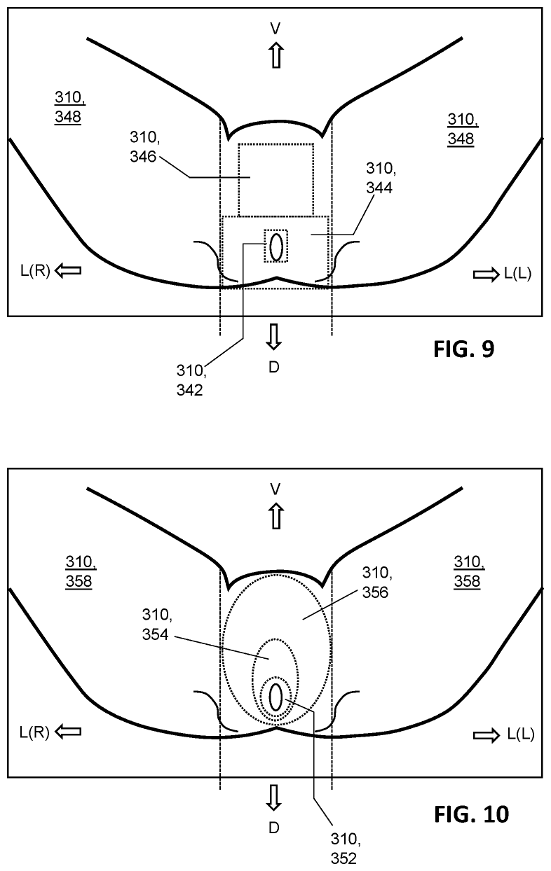

The reference illustration (300) may be superimposed with one or more zones. A zone is defined by at least one border. Each zone is identifiable using a reference such as an alphanumeric code or co-ordinates. The subject may indicate one or more zones where the sensation is felt on the reference illustration. The zone(s) corresponding to the indication(s) can be later identified using the reference(s). The size of a zone is smaller than the size of the reference illustration. Examples of reference illustrations (300) superimposed with a plurality of zones (310) are shown in FIGS. 9 to 11.

At least one zone may be located on the reference illustration (300) within a band (330). The band has a height that extends along a midline (312) of the anus (308) and genitals and having a width that extends laterally (to the left and the right) of the midline (312) of perianal and genitals. The height of the band is preferably limited by dorsal and ventral bodily edges enclosing the pelvic floor area. The width of the band is preferably limited by the lateral edges of the pelvic floor area. An example of a band (330) is shown in FIG. 8.

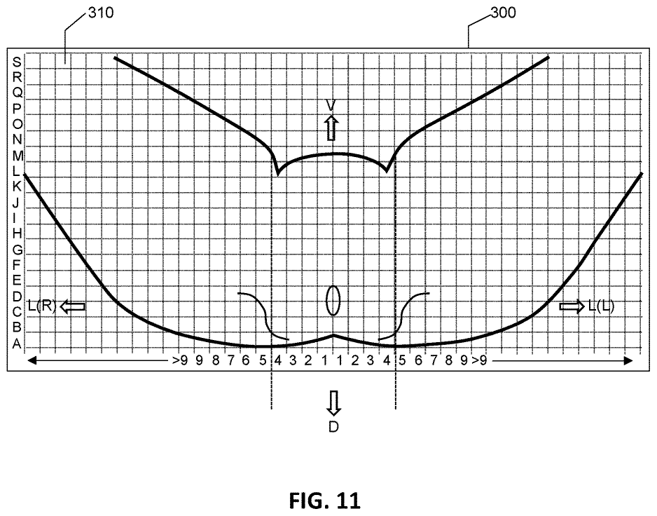

At least one zone provided on the reference illustration is an anal zone, which is disposed over at least a part of the anal region. The anal zone is defined by a border. The anal zone is located within the band where present. The anal zone preferably does not encompass or overlap with any of perianal region (212), genital region (214), and other region (218). The reference illustration may be superimposed with only one anal zone or multiple anal zones. An anal zone (342, 352) is indicated in FIGS. 9 and 10. Multiple anal zones (1-C, 1-D) are indicated in FIG. 11.

At least one zone provided on the reference illustration may be a perianal zone, which disposed over at least a part of the perianal region. The perianal zone is defined by at least one border. The anal zone is located within the band where present. The perianal zone preferably does not encompass or overlap with any of anal region (210), genital region (214), and other region (218). The reference illustration may be superimposed with only one perianal zone or multiple perianal zones. A perianal zone (344, 354) is indicated in FIGS. 9 and 10. Multiple perianal zones ((2 to 4)-(A to F), 1-(A to B), 1-(E to F)) are indicated in FIG. 11.

At least one zone provided on the reference illustration may be a genital zone, which is disposed over at least a part of the genital region. The genital zone is defined by at least one border. The anal zone is located within the band where present. The genital zone preferably does not encompass or overlap with any of anal region (210), perianal region (212), and other region (218). The reference illustration may be superimposed with only one genital zone or multiple genital zones. A genital zone (346, 356) is indicated in FIGS. 9 and 10. Multiple perianal zones ((1 to 4)-(G to L)) are indicated in FIG. 11.

At least one zone provided on the reference illustration may be an other zone, which is disposed over at least a part of the other region. The other zone is defined by at least one border. The other zone may or may not be located within the band where present.

The other zone preferably does not encompass or overlap the anal region (210). The other zone preferably does not encompass or overlap with any of anal region (210), perianal region (212). The other zone preferably does not encompass or overlap with any of anal region (210), perianal region (212), and genital region (214).

The reference illustration may be superimposed with only one other zone or multiple other zones. A other zone (348, 358) is indicated in FIGS. 9 and 10. Multiple other zones ((5 to ≥9)-(A to S); 4-(G to L)) are indicated in FIG. 11.

Preferably, the reference illustration is disposed with at least one anal zone, and with at least one other zone. Preferably, the reference illustration is disposed with at least one anal zone and at least one perianal zone and at least one other zone. Preferably, the reference illustration is disposed with at least one anal zone and at least one perianal zone and at least one genital zone and at least one other zone.

According to one zone arrangement, as exemplified, for instance, in FIGS. 9 and 10, at least some of the zones within the band are nested and have different sizes. The nested zones may have any shape e.g. ellipse-shaped (oval or circular), rectangular, regular polygonal, irregular polygonal, preferably oval. Each zone of the nested zones has a different size, and the zones are nested one within the other. The inner-most nested zone (342, 352) is preferably located at the anal region of the illustration, and subsequent nested zone (344, 354, 356) may extend ventrally (V) outwards (towards the genitals), and optionally dorsally (D) outwards and optionally laterally outwards (L). A subsequent zone (344, 354) may disposed over at least a part of the perianal region (e.g. FIGS. 9 and 10). A further subsequent zone (356) may disposed over at least a part of the genital region (e.g. FIG. 10).

According to one zone arrangement, as exemplified, for instance, in FIG. 11, at least some, preferably all of the zones are non-nested. The non-nested zones may have any shape e.g. ellipse-shaped (oval or circular), rectangular (square or oblong), regular polygonal, irregular polygonal. Each non-nested zone may have the same size or a different size. A plurality of shapes may be arranged as a grid (e.g. FIG. 11). The grid may be formed of polygons (e.g. squares) of the same size. The shapes may be arranged as patches.

At least one non-nested zone covers at least at a part of the anal region of the illustration. Preferably, at least one non-nested zone covers at least at a part of the perianal region of the illustration. Preferably, at least one non-nested zone covers at least at a part of the genital region of the illustration. Preferably, at least one non-nested zone covers at least at a part of the other region of the illustration. Preferably, at least one non-nested zone covers at least a part of pelvic floor area ventral (V) of the anal region (towards the genitals).

According to another zone arrangement, the zones are a combination of nested and non-nested zones. The illustration may contain multiple zone arrangements.

One or more indicators may be derived from the measured data of a therapeutically effective pose of the lead and/or of a therapeutically effective a set of operating parameters. By therapeutically effective, it is meant that the stimulation has a treatment effect on the pelvic floor dysfunction and/or on the pelvic floor-related disorder: side effects may also be minimised, such as cross-stimulation of other nerves.

The one or more indicators may be used to guide implantation of the lead, in particular of the electrode portion, towards a therapeutically effective pose of the lead.

The one or more indicators may be used to determine a set of operating parameters of the pulse generator that provides a therapeutically effective stimulation. The one or more indicators may be used to adjust a set of operating parameters of the pulse generator, towards a therapeutically effective stimulation.

An indicator may be a low MT. An MT that is low is indicative of a therapeutically effective pose of the lead, in particular of the electrode portion. An MT that is low is indicative of a therapeutically effective set of operating parameters, in particular, while testing different stimulation amplitude parameters. A low MT may be the lowest MT obtained while positioning the lead during implantation. Ideally a low MT is equal to or below 2 mA.

An indicator may be a ratio MT:ST<1. The parameter, ratio MT:ST, is a ratio between the measured MT and measured ST (i.e. MT/ST or ST/MT) of the subject. The units of measured MT and measured ST are the same (e.g. mA or mV). A MT:ST ratio indicating that ST is greater than MT (e.g. MT/ST<1) is indicative of a therapeutically effective position and orientation of the lead, in particular of the electrode portion. A MT:ST ratio indicating that ST is greater than MT (e.g. MT/ST<1) is indicative of a therapeutically effective set of operating parameters.

An indicator may be a high PtP-AP at ST. A PtP-AP that is high at the ST is indicative of a therapeutically effective position and orientation of the lead, in particular of the electrode portion. A PtP-AP that is high at the ST is indicative of a therapeutically effective set of operating parameters, in particular, while testing different contact configuration parameters. A high PtP-AP may be the highest PtP-AP obtained while positioning the lead during implantation. Ideally, a high PtP-AP is equal to or above 20 μV, preferably equal to or above 50 μV.

An indicator may be a high AP growth. An AP growth that is high is indicative of a therapeutically effective position and orientation of the lead, in particular of the electrode portion. An AP growth that is high is indicative of a therapeutically effective set of operating parameters, in particular, while testing different contact configuration parameters. A high AP growth may be the highest AP growth obtained while positioning the lead during implantation. The high AP growth may be indicated by a high scalar value.

Ideally a high AP growth value is ≥1.

Ideally a high AP growth gradient is >0, for a gradient (Δy/Δx) having PtP-AP as y-axis and stimulation amplitude as x-axis.

Ideally a high initial AP growth gradient is ≥0.4, more preferably ≥0.5, for a gradient (Δy/Δx) having PtP-AP as y-axis and stimulation amplitude as x-axis, and PtP-AP measured in μV and stimulation amplitude measured in mA. It is understood that different units may be used to express PtP-AP (e.g. μV, mV) and stimulation amplitude (e.g. μV, mV, μA or mA), and a threshold considered as high initial AP growth gradient can be extrapolated from PtP-AP (μV) and stimulation amplitude (mA) according to the skilled of the person in the art.

An AP growth curve that is more positively displaced from x-axis (i.e. values of PtPAP along the y-axis are higher) is indicative of a more therapeutically effective position and orientation of the lead and/or a more therapeutically effective set of operating parameters.

An indicator may be an LoS located within the anal or perianal region or genital region. LoS contained within the anal or perianal region or genital region, is indicative of a therapeutically effective position and orientation of the lead, in particular of the electrode portion. LoS contained within the anal or perianal region or genital region, is indicative of a therapeutically effective set of operating parameters.

Any of one or combination of indicators is indicative of a therapeutically effective position and orientation of the lead. The indicators may be ranked according to strength of indication, a ranking of 1 referring to the strongest indicator. Set on in Tables 1 and 2 are indicator rankings where the subject is conscious (Table 1) and unconscious (Table 2).

TABLE 1
ranking in order of strength (1 is strongest) of
indicators when the subject is conscious.
Subject is conscious

| | | Value for |
|---|---|---|
| | | therapeutically |
| Ranking | Indicator | effectiveness |
| 1 | LOS | Sensation felt in anal |
| | | or perianal region or |
| | | genital region |
| 2 | MT | Low |
| 3 | Ratio MT:ST | ST is greater than MT |
| 4 | PtP-AP | High |
| 5 | AP growth | High |

For a conscious subject, preferably the measured data comprises at least the LoS data and the MT data. For a conscious subject, more preferably the measured data comprises at least the LoS data, the MT data, and the ST data. For a conscious subject, even more preferably the measured data comprises at least the LoS data, the MT data, the ST data, and AP growth data.

TABLE 2
ranking in order of strength (1 is strongest) of
indicators when the subject is unconscious.
Subject is unconscious

| | | Value for |
|---|---|---|
| | | therapeutically |
| Ranking | Indicator | effectiveness |
| 1 | MT | Low |
| 2 | PtP-AP | High |
| 3 | AP growth | High |

For an unconscious subject, preferably the measured data comprises at least the MT data. For an unconscious subject, more preferably the measured data comprises at least the MT data and PtP-AP data. For an unconscious subject, even more preferably the measured data comprises at least the MT data, the PtP-AP data, and the AP growth data.

Pelvic floor dysfunction is understood in the art as a condition wherein a subject is unable to correctly relax and coordinate the muscles in the pelvic floor. The consequence of pelvic floor dysfunction is a pelvic floor-related disorder which includes one or more of an overactive bladder, a non-obstructive urinary retention, a faecal incontinence, pain (e.g. pelvic, rectal, anal, bladder), a sexual dysfunction (e.g. erectile dysfunction, female sexual dysfunction).

Provided herein is system for guiding implantation of an electrode portion (126) of a lead (120) of a neurostimulator for treatment of pelvic floor dysfunction and/or of a pelvic floor-related disorder of a subject, the system comprising a processor wherein the processor is configured to:
receive measured data of the subject comprising:
   pelvic floor muscle electromyography, PFM-EMG data of the subject, wherein the PFM-EMG data comprises one or more of:
   comprises:
      a) the AP growth data;
      optionally b) the MT data; a
      optionally c) the PtP-AP data; and
   optionally d) the ST data;
   optionally e) the LoS data;
determining from the measured data, real time guidance information for guiding a position and orientation of the electrode portion (126) of the lead (120) during implantation.

Provided herein is a system for guiding implantation of an electrode portion (126) of a lead (120) of a neurostimulator for treatment of pelvic floor dysfunction and/or of a pelvic floor-related disorder of a subject, the system comprising a processor wherein the processor is configured to:
receive measured data of the subject comprising:
   a) the ST data;
   b) the LoS data;
   pelvic floor muscle electromyography, PFM-EMG data of the subject, wherein the PFM-EMG data comprises one or more of:
   comprises:
      c) the AP growth data;
      optionally d) the MT data;
      optionally e) the PtP-AP data; and

The guiding is preferably towards a therapeutically effective pose of the lead. The guiding refers to pose (position and orientation) guidance during implantation. The practitioner is able to use the indicators mentioned elsewhere herein to guide the position and orientation of the electrode portion (126) towards a therapeutically-effective pose. For instance, an implantation trajectory of the electrode portion (126) which causes a lowering of the MT, may be continued until the MT reaches a minimum.

Provided herein is a system for adjusting and/or determining a set of operating parameters of an pulse generator for treatment of a pelvic floor dysfunction and/or a pelvic floor-related disorder in a subject, the system comprising a processor wherein the processor is configured to:
receive measured data of the subject comprising:
   pelvic floor muscle electromyography, PFM-EMG data of the subject, wherein the PFM-EMG data comprises one or more of:
      a) the AP growth data;
      b) optionally MT data
      c) optionally PtP-AP data
   optionally d) the ST data;
   optionally e) the LoS data;
determine, from the measured data, the set of operating parameters of the pulse generator.

The set of operating parameters of the pulse generator (160) determined is preferably therapeutically effective. The adjusting of the set of operating parameters is preferably towards a therapeutically effective set of operating parameters.

The set of operating parameters may be determined and/or adjusted during lead implantation. The set of operating parameters may be determined and/or adjusted for a pre-implanted neurostimulator. The set of operating parameters may be determined and/or adjusted for a pre-implanted neurostimulator after implantation (e.g. a first time set up and optimisation). The set of operating parameters may be determined and/or adjusted for a pre-implanted neurostimulator in response to a decrease in efficacy (e.g. some time after implantation (e.g. months or years), and after the neurostimulator had previously higher efficacy).

The set of operating parameters includes one or more of:
contact configuration parameter, or a selection of contacts in the electrode portion (126)
a stimulation amplitude parameter.

It is understood that neurostimulator may use other operating parameters such as
a frequency parameter;
a pulse width parameter.

These other parameters are typically set by the stimulation program.

Preferably set of operating parameters comprises at least a contact configuration parameter and optionally a stimulation amplitude parameter.

The contact configuration parameter is an indication a selection of electrode contacts from a plurality (e.g. 4 to 8) of contacts of a multi-contact lead. The selection is typically of the stimulation electrode of the electrode pair and reference electrode of the electrode pair from the plurality of contacts of the electrode portion (126) of the lead. Where the pulse generator housing is the reference electrode, the selection is typically of the stimulation electrode of the electrode pair from the plurality of contacts of the electrode portion (126) of the lead.

The practitioner is able to use one or more of the indicators mentioned elsewhere herein to determine a contact configuration parameter that is therapeutically-effective. The contact configuration parameter is preferably determined from the LoS data. An LoS located within the anal or perianal region or genital region is indicative of a therapeutically effective contact configuration parameter

The contact configuration parameter may be determined for a pre-implanted neurostimulator after implantation (e.g. a first time set up and optimisation). The contact configuration parameter may be determined and/or adjusted for a pre-implanted neurostimulator in response to a decrease in efficacy (e.g. some time after implantation (e.g. months or years), and after the neurostimulator had previously higher efficacy).

The stimulation amplitude parameter is an indication of a maximum amplitude of the electrical stimulation. A stimulation amplitude parameter that is therapeutically effective may be determined from the MT and ST. The maximum amplitude of the electrical stimulation is typically equal to or above the MT. The maximum amplitude of the electrical stimulation is typically equal to or below the ST.

The stimulation amplitude parameter may be determined for a pre-implanted neurostimulator after implantation (e.g. a first time set up and optimisation). The stimulation amplitude parameter may be determined and/or adjusted for a pre-implanted neurostimulator in response to a decrease in efficacy (e.g. some time after implantation (e.g. months or years), and after the neurostimulator had previously higher efficacy).

A stimulation frequency parameter is an indication of a frequency of the electrical stimulation. A stimulation frequency parameter that is therapeutically effective is typically between 2 and 40 Hz.

A pulse width parameter is an indication of a pulse width of the electrical stimulation. A pulse width parameter that is therapeutically effective is typically between 80 and 150 msec.

The practitioner is able to use one or more of the indicators mentioned elsewhere herein to determine a set of operating parameters that is therapeutically-effective.

The practitioner is able to use one or more of the indicators mentioned elsewhere herein to guide adjustment to a set of operating parameters that is therapeutically-effective. The adjustment may be made after implantation after implantation (e.g. a first time set up and optimisation). The adjustment may be made to a pre-implanted neurostimulator in response to a decrease in efficacy (e.g. some time after implantation, and after the neurostimulator had a previously higher efficacy).

Where an adjustment to an operating parameter causes an indicator to move towards value that is a therapeutically effective, a direction of the adjustment may be continued until a value of the indicator is maximally within the range for therapeutic effectiveness.

The processor configured to carrying out the presently-described method may be any type of processor, capable of executing the steps described herein.

The processor may be a digital signal processor (DSP), a microcontroller, a field programmable gate array (FPGA), an application specific integrated circuit (ASIC), etc.).

The processor may be comprised in a standard computer system such as an Intel Architecture IA-32 based computer system 2, and implemented as programming instructions of one or more software modules stored on non-volatile (e.g., hard disk or solid-state drive) storage associated with the corresponding computer system. However, it will be apparent that at least some of the steps of any of the described processes could alternatively be implemented, either in part or in its entirety, as one or more dedicated hardware components, such as gate configuration data for one or more field programmable gate arrays (FPGAs), or as application-specific integrated circuits (ASICs), for example.

The processor is preferably external and not implantable. The processor is preferably not a part of the neurostimulator. It is within the scope of the invention that the processor is incorporated into the neurostimulator.

The system may include a device for performing the pelvic floor muscle electromyography on the subject.

Further provided is a computer program comprising instructions which, when the program is executed by a computer, cause the computer to carry out the steps recited herein.

Further provided is a computer-readable medium comprising instructions which, when the program is executed by a computer, cause the computer to carry out the steps recited herein.

Further provided is a computer-implemented method, wherein the method comprises the steps carried out by the processor described herein.
Provided herein is a method for guiding a practitioner towards a therapeutically effective pose of the lead during implantation, comprising: adjusting the pose of the lead until one or more of the indicators in Table 1 reaches the value for therapeutically effectiveness.

Provided herein is a method for guiding a practitioner towards a therapeutically effective pose of the lead during implantation, comprising:
adjusting the pose of the lead until one or more of the indicators in Table 2 reaches the value for therapeutically effectiveness.

The indicator may be selected according to its ranking.

### Example

64 female patients each presented with an overactive bladder and/or non-obstructive urinary retention. Each was treated by neuromodulation, wherein a neurostimulator was implanted, and electrode contacts of the lead were positioned adjacent to the sacral nerve or pudendal root of the subject. After recovery, each patent was assessed for success or failure of the treatment. Indicators of success or failure included an absence or presence of leakage. AP growth was measured in each female patient using an PFM-EMG probe inserted intravaginally. The PFM-EMG probed was a MAPLe probe having 12 areas represented by 12 (bipolar) electrodes. The MAPLe probe was divided in 4 quadrants corresponding to the different sides of the pelvic floor (clockwise starting at 12 o'clock: anterior, left/contralateral, posterior, right/ipsilateral). Each side was divided in 3 crescent-shaped fields corresponding with the different depths (from the inner to the outer circles: deep, center, superficial).

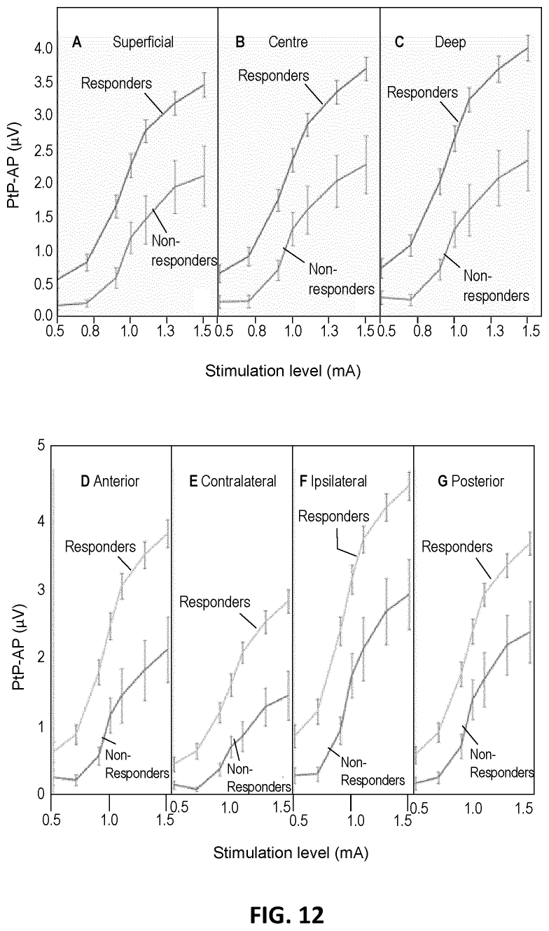

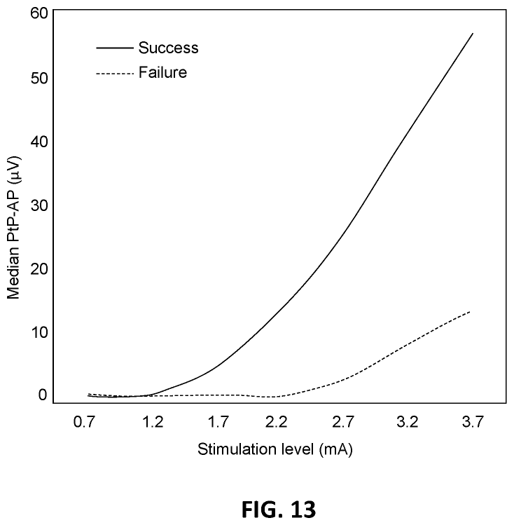

FIG. 12 illustrates AP growth curves of the group of patients that failed (non-responders) versus the success group (responders), for the separate PFM-EMG probe areas (A: superficial depth; B: center depth; C: deep depth D: anterior side; E: contralateral side; F: ipsilateral side and G: posterior side). The relative stimulation intensities delivered by the lead contacts are presented on the x-axis, the measured peak-to-peak action potential (PtP-AP) is presented on the x-axis. Sensory threshold (ST) was 0.7 mA. The mean PtP-AP of the Responders (R) and non-responders (NR) are seen for the different sides and depths of the MAPLe probe. FIG. 13 illustrates AP growth curves of the group of patients that failed (dotted line) versus the success group (solid line), where all locations of FIG. 12 have been combined and the median value is shown with error bars.

Based upon outcome (responders vs non-responders), each and every depth (A: superficial (MMA: p=0.0178), B: center (MMA: p=0.0142), C: deep (MMA: p=0.0048)) and each and every side (D: anterior (MMA: p=0.0031), E: contralateral (MMA: p=0.0019), F: ipsilateral (MMA: p=0.0426) and G: posterior (MMA: p=0.0398)) presented with significantly different regression coefficients, indicating responders present with higher gradients compared to non-responders. Each error bar is constructed using 1 standard error from the mean. MMA means mixed model analysis as a statistical test.

The results demonstrate clear non-overlapping curves between responders and non-responders, indicative of a high discriminatory power of AP growth.

1. A system for guiding implantation of an electrode portion of a lead of a neurostimulator for treatment of a pelvic floor dysfunction and/or of a pelvic floor-related disorder of a subject, the system comprising a processor, wherein the processor is configured to:
receive measured data of the subject comprising:
pelvic floor muscle electromyography (PFM-EMG) data of the subject, wherein the PFM-EMG data comprises:
a) action potential (AP) growth data, comprising one or more measurements of the peak-to-peak action potential growth (AP growth) of the subject, wherein the AP growth is an indication of an increase in contraction force per unit of stimulation delivered by the pulse generator;
and optionally b) motor threshold (MT) data comprising one or more measurements of the motor threshold of the subject, where the MT is an indication of a minimum stimulation amplitude delivered by a pulse generator of the neurostimulator that induces a measurable action potential in the pelvic floor of the subject as measured by the PFM-EMG;
and optionally c) peak-to-peak amplitude of the action potential (PtP-AP) data comprising one or more measurements of the PtP-AP of the subject, wherein the PtP-AP is an indication of a contraction force of the pelvic floor as measured by the PFM-EMG during stimulation delivered by the pulse generator;
wherein the system determines from the measured data real time guidance information for guiding a position and/or orientation of an electrode portion during implantation.

2. A system for adjusting and/or determining a set of operating parameters of a pulse generator of a neurostimulator for treatment of a pelvic floor dysfunction and/or of a pelvic floor-related disorder in a subject, the system comprising a processor, wherein the processor is configured to:
receive measured data of the subject comprising:
pelvic floor muscle electromyography (PFM-EMG) data of the subject, wherein the PFM-EMG data comprises:
a) action potential (AP) growth data comprising one or more measurements of the peak-to-peak action potential growth (AP growth) of the subject, wherein the AP growth is an indication of an increase in contraction force per unit of stimulation delivered by the pulse generator;
and optionally b) motor threshold (MT) data comprising one or more measurements of the motor threshold of the subject, where the MT is an indication of a minimum stimulation amplitude delivered by a pulse generator of the neurostimulator that induces a measurable action potential in the pelvic floor of the subject as measured by the PFM-EMG;
and optionally c) peak-to-peak amplitude of the action potential (PtP-AP) data comprising one or more measurements of the PtP-AP of the subject, wherein the PtP-AP is an indication of a contraction force of the pelvic floor as measured by the PFM-EMG during stimulation delivered by the pulse generator;
wherein the system determines from the measured data a set of operating parameters of the pulse generator.

3. The system according to claim 1, wherein
an MT that is lowest is indicative of a therapeutically effective pose of the electrode portion.

4. The system according to claim 1, wherein
a PtP-AP that is highest is indicative of a therapeutically effective pose of the electrode portion.

5. The system according to claim 1, wherein
an AP growth that is highest is indicative of a therapeutically effective pose of the electrode portion.

6. The system according to claim 1, wherein the measured data further comprises self-observation data of the subject, wherein the self-observation data comprises:
d) sensory threshold (ST) data comprising one or more measurements of the ST of the subject, wherein the ST is an indication of a minimum stimulation amplitude delivered by the pulse generator that induces a physical sensation felt by the subject; and
e) location of sensation (LoS) data comprising one or measurements of the LoS of the subject data, wherein the LoS is an indication by the subject of a location of the feeling of the physical sensation in the body caused by stimulation delivered by the pulse generator.

7. The system according to claim 6, wherein a MT:ST ratio indicating that ST is greater than MT is indicative of a therapeutically effective position and orientation of the electrode portion.

8. The system according to claim 6, wherein a LoS contained within an anal region or perianal region or genital region of the subject is indicative of a therapeutically effective position and orientation of the electrode portion.

9. The system according to claim 6, wherein the d) ST data and the e) LoS data are collected while the subject is conscious.

10. The system according to claim 1, wherein the a) AP growth data, optionally the b) MT data and optionally the c) PtP-AP data are collected while the subject is conscious or unconscious.

11. The system according to claim 6, wherein the measured data comprises a) the AP growth data, b) the MT data, c) the PtP-AP data, d) the ST and e) the LoS data.

12. The system according to claim 1, wherein the measured data is collected while the electrode portion of the lead is being positioned towards or is placed adjacent to a sacral or pudendal root of the subject.

13. The system according to claim 1, wherein the pelvic floor-related disorder is one or more of an overactive bladder, non-obstructive urinary retention, faecal incontinence, pain, and sexual dysfunction.

14. A computer program comprising instructions which, when the program is executed by a computer, cause the computer to carry out the steps recited in claim 1.

15. A computer-readable medium comprising instructions which, when the program is executed by a computer, cause the computer to carry out the steps recited in claim 1.

16. The system according to claim 2, wherein
an MT that is lowest is indicative of a therapeutically effective set of operating parameters.

17. The system according to claim 2, wherein
a PtP-AP that is highest is indicative of a therapeutically effective set of operating parameters.

18. The system according to claim 2, wherein
an AP growth that is highest is indicative of a therapeutically effective set of operating parameters.

19. The system according to claim 2, wherein the measured data further comprises self-observation data of the subject, wherein the self-observation data comprises:
d) sensory threshold (ST) data comprising one or more measurements of the ST of the subject, wherein the ST is an indication of a minimum stimulation amplitude delivered by the pulse generator that induces a physical sensation felt by the subject; and
e) location of sensation (LoS) data comprising one or measurements of the LoS of the subject data, wherein the LoS is an indication by the subject of a location of the feeling of the physical sensation in the body caused by stimulation delivered by the pulse generator.

20. The system according to claim 19, wherein a MT:ST ratio indicating that ST is greater than MT is indicative of a therapeutically effective set of operating parameters.
